(19) United States
(12) Patent Application Publication
(10) Pub. No.: US 2022/0040011 A1
(43) Pub. Date: Feb. 10, 2022

(54) SELF-ADHESIVE ABSORBENT HYGIENIC PATCH WORN ON THE BODY

(71) Applicants: Alison NADEL, Arlington, VA (US); Julianne WEINER, Boyds, MD (US)

(72) Inventors: Alison NADEL, Arlington, VA (US); Julianne WEINER, Boyds, MD (US)

(21) Appl. No.: 17/392,930

(22) Filed: Aug. 3, 2021

Related U.S. Application Data
(60) Provisional application No. 63/061,034, filed on Aug. 4, 2020.

(51) Int. Cl. A61F 13/53; A61F 13/56

(52) U.S. Cl. CPC A61F 13/53; A61F 2013/53035; A61F 13/56

(57) ABSTRACT

A self-adhesive absorbent patch includes an absorbent layer that is an outermost layer when the self-adhesive absorbent patch is applied to a user's body and/or garment and absorbs liquids, droplets, and/or moisture expelled by the user, a resistant or impermeable layer that is bonded to the first layer and prevents or limits the liquids, droplets, and/or moisture absorbed from the absorbent layer from being deposited onto the user's body and/or garment, an adhesive layer comprised of a double-sided adhesive, wherein a first side of the double-sided adhesive adheres to the resistant or impermeable layer and a second side of the double-sided adhesive adheres to the user's body and/or garment when the user applies the self-adhesive absorbent patch to the user's body and/or garment, and a release liner that is removably attached to the adhesive layer before the user applies the self-adhesive absorbent patch to the user's body and/or garment.

### CROSS REFERENCED RELATED APPLICATIONS

This application claims priority to U.S. Provisional Patent Application No. 63/061,034, filed on Aug. 4, 2020, the entirety of which is incorporated herein by reference.

### BACKGROUND OF THE INVENTION

Preventing the spread of germs has taken on new and more urgent importance due to the COVID-19 pandemic. Public health professionals have urged the public to cover coughs and sneezes, either with a tissue or in the crook of the elbow. For example, the Centers for Disease Control and Prevention recommends “Cover your mouth and nose with a tissue when you cough or sneeze,” and “If you don't have a tissue, cough or sneeze into your elbow, not your hands.” See CDC website on Coughing and Sneezing, available at https://www.cdc.gov/healthywater/hygiene/etiquette/coughing_sneezing.html. Accessed Aug. 2, 2021.

However, tissues are not always readily available when they are needed to catch a cough or sneeze. Indeed, even a tissue box within arm's reach may not be accessible in time for the sudden cough or sneeze. In addition, many people—including children, who have not yet learned such hygienic practices or formed the habit of using them—do not remember to direct their coughs and sneezes into their elbows.

There is a need for a readily accessible, absorbent product that attaches to the body, readily available and accessible when needed to catch coughs and sneezes and to wipe the face as needed. There is also a need for a visual reminder to teach and ingrain the habit of directing coughs and sneezes to certain locations on the body that are most likely to catch coughs and sneezes, including for example, the elbow. These and other drawbacks exist.

### BRIEF DESCRIPTION OF THE DRAWINGS

FIG. 1 is a view of an example of the layers of an example of a self-adhering absorbent patch.

FIG. 2A illustrates a cross-sectional view of an example of a self-adhering absorbent patch.

FIG. 2B illustrates a cross-sectional view of an example of a self-adhering absorbent patch.

FIG. 2C illustrates a cross-sectional view of an example of a self-adhering absorbent patch.

FIG. 2D illustrates a cross-sectional view of an example of a self-adhering absorbent patch.

FIG. 3A illustrates an example of a self-adhering absorbent patch placed on the arm.

FIG. 3B illustrates an example of a self-adhering absorbent patch placed on the arm.

FIG. 3C illustrates an example of a self-adhering absorbent patch placed on the wrist.

FIG. 3D illustrates an example of a self-adhering absorbent patch placed on the arm.

FIG. 4A illustrates an example of an adhesive pattern on a face of a self-adhering absorbent patch that adheres to the body.

FIG. 4B illustrates an example of an adhesive pattern on a face of a self-adhering absorbent patch that adheres to the body.

FIG. 4C illustrates an example of an adhesive pattern on a face of a self-adhering absorbent patch that adheres to the body.

FIG. 4D illustrates an example of an adhesive pattern on a face of a self-adhering absorbent patch that adheres to the body.

FIG. 4E illustrates an example of an adhesive pattern on a face of a self-adhering absorbent patch that adheres to the body.

FIG. 4F illustrates an example of an adhesive pattern on a face of a self-adhering absorbent patch that adheres to the body.

FIG. 4G illustrates an example of an adhesive pattern on a face of a self-adhering absorbent patch that adheres to the body.

FIG. 4H illustrates an example of an adhesive pattern on a face of a self-adhering absorbent patch that adheres to the body.

FIG. 4I illustrates an example of an adhesive pattern on a face of a self-adhering absorbent patch that adheres to the body.

FIG. 4J illustrates an example of an adhesive pattern on a face of a self-adhering absorbent patch that adheres to the body.

FIG. 4K illustrates an example of an adhesive pattern on a face of a self-adhering absorbent patch that adheres to the body.

### DETAILED DESCRIPTIONS OF PREFERRED EMBODIMENTS

Hygienic practices recommend covering coughs and sneezes by directing the mouth and nose into the elbow or directly into a tissue, when available. According to various examples, an absorbent patch worn on the body and/or a garment provides a readily accessible alternative to a loose tissue. Adhesion to a user's body, or similarly to a user's garment, ensures that it is accessible when needed, which may be unexpected, as in the case of a sudden sneeze or cough. The various examples described herein also serve as a visual reminder to users where to direct their coughs and sneezes. A self-adhesive absorbent patch according to the various examples can catch and absorb droplets from the mouth, nose, eyes, and/or face, and the self-adhesive absorbent patch can then be safely and easily removed and discarded after one or more sneezes or coughs, rather than remaining on a user's arm.

A self-adhesive absorbent patch, according to the various examples shown and described herein, would address these and other needs. Among other benefits, a self-adhesive absorbent patch: (1) is readily accessible; (2) provides a visual reminder to users to use the crook of their elbow to cover coughs and sneezes; and (3) catches and absorbs droplets that would otherwise land on the arm or other part of the body, allowing for them to be safely and easily removed and easily discarded. It may be particularly useful to help ingrain the habit of directing coughs and sneezes in the recommended place for children and others who are learning hygienic practices. A self-adhesive absorbent patch according to the various examples shown and described herein may be placed in, for example, the elbow or crook of a person's arm, where it can serve as a reminder where to cough and sneeze. A self-adhesive absorbent patch according to the various examples shown and described herein may also be placed on or near the wrist, where it can be used to wipe a user's nose, mouth, eyes, and/or face as needed. A self-adhesive absorbent patch according to the various examples shown and described herein could be changed and/or replaced regularly as needed.

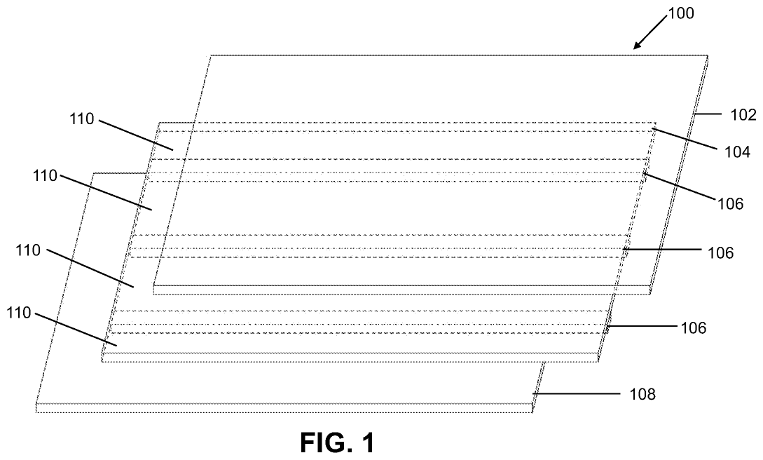

A self-adhesive absorbent patch may be constructed in various sizes, shapes, and configurations. FIG. 1 shows example layers in an example of a self-adhesive absorbent patch 100.

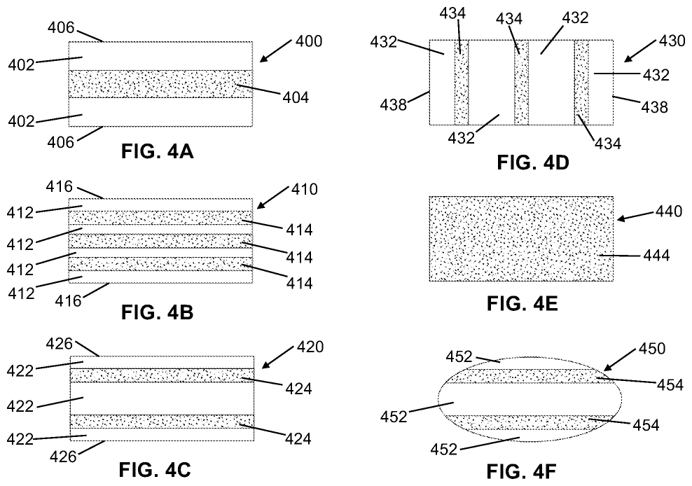

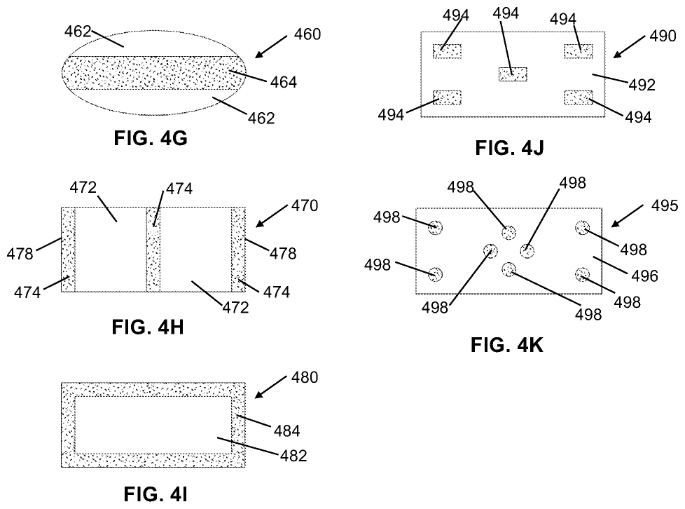

The shape and size of the self-adhesive absorbent patch may vary based on the size and age of the user, the placement of the self-adhesive absorbent patch on the user's body or garment, and/or the method of manufacture. For example, the shape may be a rectangle, square, oval, or other shape. The shape of the self-adhesive absorbent patch may be a polygon or non-polygon. It may be symmetrical or asymmetrical. The shape also may be one that is most effective for fitting to and/or adhering to a certain location of the user's body. FIGS. 4A, 4B, 4C, 4D, 4E, 4H, 4I, 4J, and 4K are examples of rectangular shapes that may be used. FIGS. 4F and 4G are examples of oval shapes that may be used. Other examples of shapes and sizes may be used.

A smaller self-adhesive absorbent patch, for example, may be used on young children, who generally have smaller arms, than on adolescents and adults who generally have larger arms. Additionally, the self-adhesive absorbent patch intended to be placed on an arm, for example, may be a different size and shape than that intended to be placed on a wrist, given the different sizes and shapes of those body parts and the movement used in order to bring those body parts in contact with or in proximity to a user's face. Additionally, the size and shape may vary based on the intended activity of the person while using the self-adhesive absorbent patch. For example, the size of a self-adhesive patch applied to bulky outerwear such as a parka during an outdoor winter activity may be larger, than, for example, the size of a self-adhesive patch applied to a close-fitting long-sleeve shirt worn indoors.

In addition to variations in size and shape, there are also examples of different configurations of a self-adhesive absorbent patch.

One example may be a self-adhesive absorbent patch with a four-layer configuration. In this configuration, the top or outermost layer sits farthest from the body when applied. The top layer may be an absorbent material that may absorb liquids, droplets, and/or moisture. The top layer may be flexible and/or pliable, and it may feel soft to the touch. The top layer may be a woven or nonwoven material. It may be made of paper, tissue, fabric, and/or other fibers or materials. Examples of materials in the top layer may include cotton, cellulose, polyester, viscose, and/or other natural and/or synthetic fibers. The material in the top layer may be suitable for use in hygienic and/or medical applications. The material in the top layer may be considered hypoallergenic, non-abrasive, and suitable for contact with human skin. An example of the function of this layer is that it may absorb any saliva, mucous, tears, sweat, and/or other fluids coming from the mouth, nose, eyes and/or face, and feels comfortable to the touch to a reasonable user. The material in the top layer may be printable with patterns, designs, characters, and/or text, which may function to cause users to direct their faces to the self-adhesive absorbent patch when, for example, coughing, sneezing, and/or wiping their faces, and may also function to aesthetically appeal to users, including children.

A second layer in a four-layer configuration may be a fluid, liquid, and/or moisture resistant and/or impermeable layer. Examples of the material or materials comprising this layer may be polyurethane, polyethylene, and/or materials with similar properties. The second layer may be thin and flexible. The top layer and the second layer in a four-layer configuration may be bonded and/or adhered and/or laminated together. An example of a function of this layer is that it may resist or prevent saliva, mucous, tears, sweat, and/or other fluids being expelled by the mouth, nose, eyes and/or face from penetrating through the self-adhesive absorbent patch onto a user and/or a user's garment. Another example of a function of the second layer in a four-layer configuration is that it may enhance the structural integrity of the self-adhesive absorbent patch, allowing it to be safely and easily removed from a user's body or garment without ripping and/or tearing.

A third layer in a four-layer configuration may be an adhesive layer. An adhesive may, for example, be suitable and safe for use on human skin as well as a variety of fabrics. An adhesive may, for example, be removed from skin and garments without leaving a residue and without removing body hair from a user, if applied directly to the skin. An adhesive may, for example, be comprised of acrylic, acrylate, silicon, and/or another suitable material. An adhesive may, for example, be pressure sensitive and suitable for use in hygienic and/or medical applications. An adhesive layer may be double-coated and/or have double-sided adhesion, allowing it to adhere to the layers above and below it. An adhesive layer may have a fluid, liquid, and/or moisture resistant backing or carrier.

An example of a function of an adhesive layer is to allow the self-adhesive absorbent patch to adhere to a user and/or a user's garment. In the example of an adhesive with double-sided adhesion, one side may adhere to the layer above it, and the other side may adhere to user and/or a user's garment, after removal of a release liner. Another example of a function of an adhesive layer may be to make a self-adhesive absorbent patch more resistant to fluids, liquids, and/or moisture, resisting or preventing penetration through to a user and/or a user's garment. Another example of a function of an adhesive later may be to improve the structural integrity of a self-adhesive absorbent patch. Another example of a function of an adhesive layer may be to improve ease of removing the self-adhesive absorbent patch without it ripping and/or tearing.

An adhesive layer may be applied in different configurations. For example, the adhesive layer may be applied in a solid layer to an entire side of the self-adhesive absorbent patch. An example of this application is shown in FIG. 4E. An adhesive layer may also be applied around the perimeter of the self-adhesive absorbent patch. An example of this application is shown in FIG. 4I. An adhesive layer may also be applied in parallel strips parallel to a longer side of a self-adhesive absorbent patch. Examples of this application are shown in FIGS. 4A, 4B, 4C, and 4F. An adhesive layer may also be applied in parallel strips parallel to a shorter side of a self-adhesive absorbent patch. An example of this application is shown in FIGS. 4D and 4H. An adhesive layer may also be applied in spots or patches on a self-adhesive absorbent patch. Such spots or patches may be round or rectangular or square. Examples of these applications are shown in FIGS. 4J and 4K.

The fourth layer in a four-layer configuration may be a release liner. The fourth layer in a four-layer configuration would, for example, cover and removably adhere to the adhesive layer prior to application. When a user wishes to use and/or apply the self-adhesive absorbent patch, a release liner would be peeled away from an adhesive layer, exposing the adhesive layer. Once the release layer is removed, a self-adhesive patch may then be applied with the adhesive layer placed upon user and/or user's garment. The release liner may then be discarded.

Another example of a configuration may be a self-adhesive absorbent patch with a three-layer configuration. In an example of a three-layer configuration, the top or outermost layer sits farthest from the body when applied. Similar to the top layer described in the example four-layer configuration described above, an example of a top layer in a three-layer configuration may be an absorbent material that may absorb liquids, droplets, and/or moisture. A top layer in a three-layer configuration may have similar characteristics to the top layer in the example four-layer configuration described above, with respect to absorbency, flexibility, pliability, and softness. The material in a top layer may be printable with patterns, designs, characters, and/or text, which may function to cause users to direct their faces to the self-adhesive absorbent patch when, for example, coughing, sneezing, and/or wiping their faces, and may also function to aesthetically appeal to users, including children.

An example of the function of the top layer in a three-layer configuration is that it may absorb any saliva, mucous, tears, sweat, and/or other fluids coming from the mouth, nose, eyes and/or face, and feels comfortable to the touch to a reasonable user. Another example of the function of the top layer in a three-layer configuration is that it may be sufficiently thick and/or absorbent to resist or prevent liquid, droplets, and/or moisture from penetrating through the self-adhesive absorbent patch and onto a user and/or a user's garment. Another example of the function of the top layer in a three-layer configuration is that it may be sufficiently strong to allow a self-adhesive absorbent patch to be removed and/or pulled from a user and/or a user's garment in one piece without ripping and/or tearing. Another example of the function of the top layer in a three-layer configuration is that it may help to maintain the structural integrity of the self-adhesive absorbent patch. Another example of the function of the top layer in a three-layer configuration is that it may reduce the need and/or desirability of including an additional layer between such a top layer and an adhesive layer, such as the example of the second layer described in the four-layer configuration example described above.

Similar to the top layer in the example of a four-layer configuration described above, the top layer in a three-layer configuration may be a woven or nonwoven material. It may be made of paper, tissue, fabric, and/or other fibers or materials. Examples of materials in this layer may include cotton, cellulose, polyester, viscose, and/or other natural and/or synthetic fibers. This material may be suitable for use in hygienic and/or medical applications. This material may be considered hypoallergenic, non-abrasive, and suitable for contact with human skin.

A second layer in an example three-layer configuration may be an adhesive layer, which may be the same as or substantially similar to the adhesive layer in the four-layer configuration described above. An adhesive layer in a three-layer configuration may have a shape and size such that it provides additional structural integrity to the self-adhesive absorbent patch.

A third layer in an example three-layer configuration may be the release liner, which may be the same as or substantially similar to the release liner in the four-layer configuration described above.

When assembled, the layers in any configuration would result in a self-adhesive absorbent patch that is absorbent on the side that faces away from the body when applied and has adhesive on the side that is applied to the body. When assembled, the layers would result in a self-adhesive absorbent patch that is flexible, pliable, thin, and would not inhibit ordinary movement when applied to a user and/or a user's garment.

Referring now to the figures, FIG. 1 illustrates an example self-adhesive absorbent patch 100 having a four-layer construction according to various examples of the disclosure. Self-adhesive absorbent patch 100 may include a first layer 102, a second layer 104, a third layer 106, and a fourth layer 108. As shown in FIG. 1, self-adhesive absorbent patch 100 may include one or more empty spaces or voids 110 that may be located within and/or created by placement of, for example, the adhesive material in a third layer 106.

First layer 102 may be an absorbent material that may absorb liquids, droplets, and/or moisture. First layer 102 may be the outermost layer of self-adhesive absorbent patch 100 and face away from the body when applied to a user and/or a user's garment as shown and described herein. First layer 102 may be flexible and/or pliable, and may feel soft to the touch. First layer 102 may be a woven or nonwoven material. First layer 102 may be comprised of paper, tissue, fabric, and/or other fibers or materials. Examples of materials in first layer 102 may include cotton, cellulose, polyester, viscose, and/or other natural and/or synthetic fibers. Such material may be suitable for use in hygienic and/or medical applications. Such material may be considered hypoallergenic, non-abrasive, and suitable for contact with human skin. First layer 102 may function to absorb any saliva, mucous, tears, sweat, and/or other fluids coming from the mouth, nose, eyes and/or face, and may feel comfortable to the touch to a reasonable user.

Second layer 104 may be a fluid, liquid, and/or moisture resistant and/or impermeable layer. As shown in FIG. 1, second layer 104 may be located between first layer 102 and third layer 106 such that first layer 102 can absorb fluid, liquid, and/or moisture and third layer 106 can cause self-adhesive absorbent patch 100 to adhere to a user and/or a user's garment. Second layer 104 may be comprised of polyurethane, polyethylene, or other comparable material that resists or prevents liquids from penetrating through self-adhesive absorbent patch. Second layer 104 may be thin, such that it adds only minimal thickness to self-adhesive absorbent patch. Second layer 104 may, for example, be approximately 1 millimeter thick. In various examples, the thickness of second layer 104 may depend upon the material used in second layer 104 and/or first layer 102. Second layer 104 may also be pliable and/or flexible, such that it does not inhibit ordinary movement when, for example, applied to the arm of a user.

Second layer 104 may function to resist and/or prevent saliva, mucous, tears, sweat, and/or other fluids coming from the mouth, nose, eyes and/or face from penetrating through the self-adhesive absorbent patch onto a user and/or a user's garment. Second layer 104 may also function may function to enhance the structural integrity of the self-adhesive absorbent patch, allowing it to be removed from a user and/or a user's garment without ripping or tearing. For example, second layer 104 may provide additional strength and durability to self-adhesive absorbent patch 100 than that which first layer 102 provides on its own. Second layer 104 may also allow for a user to remove a self-adhesive absorbent patch with one hand without causing it to rip and/or tear and/or leave part of self-adhesive absorbent patch adhered to the user.

Third layer 106 may be a non-continuous layer of adhesive consisting of, for example, three strips of double-sided adhesive. Though three strips of double-sided adhesive are shown in FIG. 1, it will be appreciated that any number of adhesive strips and/or other configurations may be used to cause self-adhesive absorbent patch to adhere to a user and/or a user garment. Examples of such configurations are shown in FIGS. 4A, 4B, 4C, 4D, 4E, 4F, 4G, 4H, 4I, 4J, 4K. Other configurations, depending on the size and shape of the self-adhesive absorbent patch, for example, may be used.

Third layer 106 may be suitable and safe for use on human skin as well as a variety of fabrics. Third layer 106 may, for example, be removed from skin and garments without leaving a residue and without removing body hair from a user, if applied directly to the skin. Third layer 106 may be comprised of acrylic, acrylate, silicon, and/or another suitable material. Third layer 106 may be suitable for use in hygienic and/or medical applications. Third layer 106 may be double-coated and/or have double-sided adhesion, allowing it to adhere to the layers above and below it. An adhesive layer may have a fluid, liquid, and/or moisture resistant backing or carrier.

Third layer 106 may function to adhere self-adhesive absorbent patch 100 to a user and/or a user's garment. In the example of an adhesive with double-sided adhesion, one side may adhere to the layer above it, e.g., second layer 104, and the other side may adhere to user and/or a user's garment, after removal of fourth layer 108, which may be, for example, a release liner. Another example of a function of an adhesive layer, e.g., third layer 106, may be to make a self-adhesive absorbent patch more resistant to fluids, liquids, and/or moisture, resisting or preventing penetration through to a user and/or a user's garment. Another example of a function of an adhesive layer, e.g., third layer 106, may be to improve the structural integrity of a self-adhesive absorbent patch. Another example of a function of an adhesive layer, e.g., third layer 106, may be to improve ease of removing the self-adhesive absorbent patch without it ripping and/or tearing the thing to which self-adhesive absorbent patch 100 is applied (e.g., a user's skin or a user's garment).

First layer 102 and second layer 104 in a four-layer configuration may be bonded and/or adhered and/or laminated together. Second layer 104 and third layer 106 in a four-layer configuration may be bonded and/or adhered and/or laminated together, including, for example, by the adhesive properties of third layer 106. Third layer 106 may be lightly or gently adhered to fourth layer 108 prior to application, which may function as a release liner thereby allowing fourth layer 108 to easily separate from third layer 106 without adversely impacting the adhesive properties of third layer 106 and/or the structural integrity of self-adhesive absorbent patch 100. In this way, fourth layer 108 can effectively be removed from self-adhesive absorbent patch 100 and discarded, and the remaining three layers of self-adhesive absorbent patch 100 may be applied to a user and/or a user's garment.

Fourth layer 108 may function, for example, as a release liner that covers third layer 106 prior to application to apply to a user and/or a user's garment. When a user wishes to apply self-adhesive absorbent patch 100 to the body or a garment, fourth layer 108 may be peeled away from the third layer 106, exposing adhesive strips, for example, prior to application. As discussed above, third layer 106 may be adhered to fourth layer 108 prior to application. Fourth layer 108 may function as a release liner thereby allowing fourth layer 108 to easily separate from third layer 106 without adversely impacting the adhesive properties of third layer 106 and/or the structural integrity of self-adhesive absorbent patch 100. In this way, fourth layer 108 can effectively be removed from self-adhesive absorbent patch 100 and discarded, and the remaining three layers of self-adhesive absorbent patch 100 may be applied to a user and/or a user's garment.

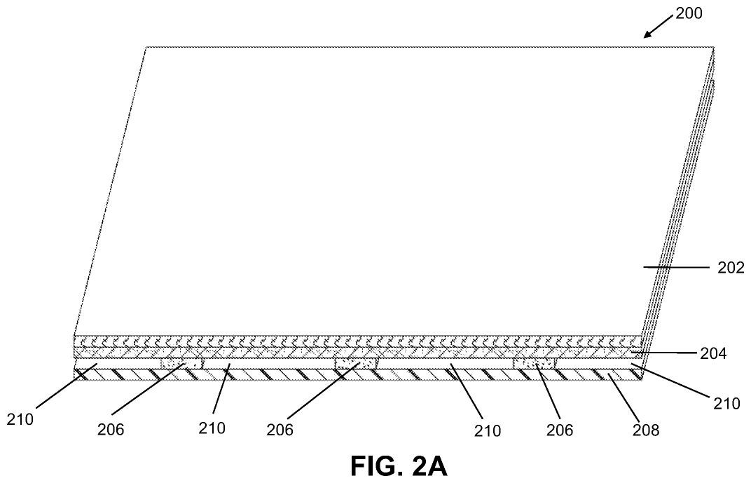

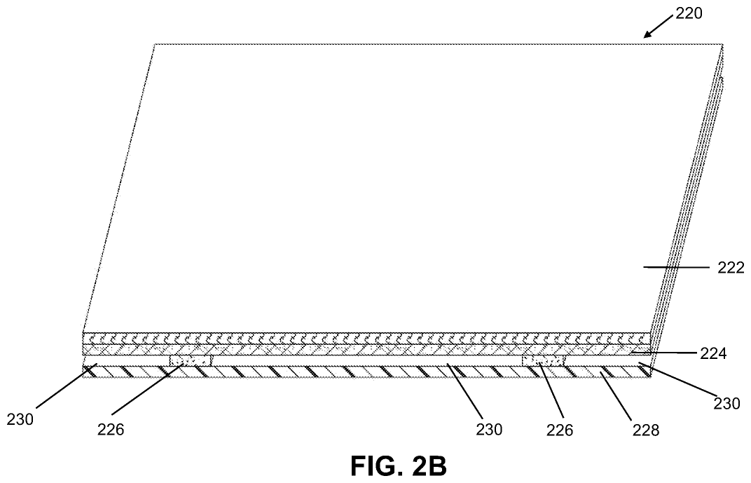

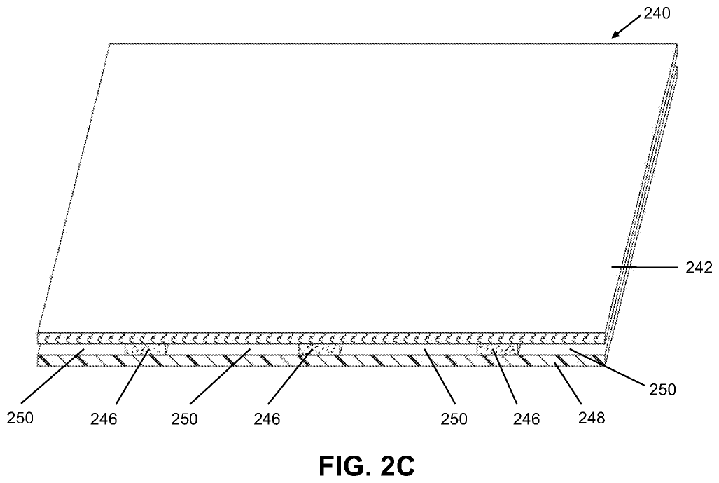

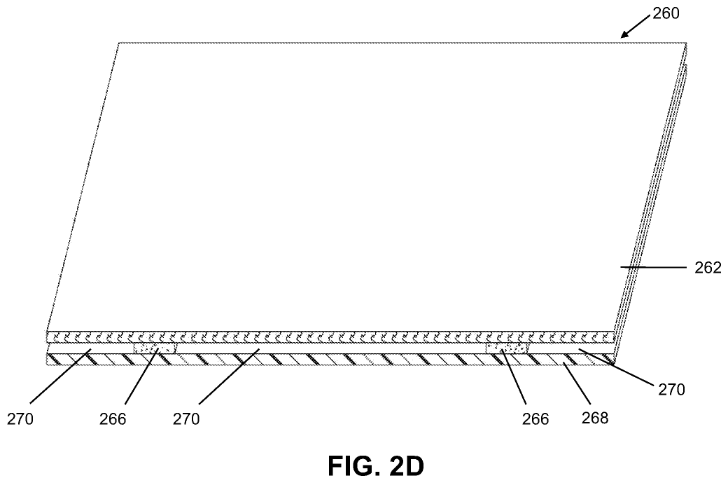

FIGS. 2A and 2B illustrate cross-sectional views of examples of self-adhesive absorbent patches 200, 220, respectively, having a four-layer configuration. FIG. 2A illustrates a cross sectional view of a self-adhesive absorbent patch 200 with a four-layer configuration and three strips of adhesive in the third layer. FIG. 2B illustrates a cross sectional view of a self-adhesive absorbent patch 220 with a four-layer configuration and two strips of adhesive in the third layer.

FIG. 2A illustrates a cross-sectional view of an example of a self-adhesive absorbent patch 200 having a four-layer configuration and three strips of adhesive in third layer 206. First layer 202 may face away from the body when applied to a user, meaning that first layer 202 is the outermost layer that is farthest from a user's arm when applied, for example, to the arm. This allows first layer 202 to be moved towards a user's face when, for example, the user uses self-adhesive absorbent patch to catch a cough or sneeze. First layer 202 may be the same or similar to first layer 102 as shown and described in FIG. 1. First layer 202 may be bonded and/or adhered and/or laminated to a second layer 204 that may be a fluid, liquid, and/or moisture resistant and/or impermeable layer similar to second layer 104 as shown and described in FIG. 1. Third layer 206 may be a non-continuous layer of adhesive consisting of, for example, three strips of adhesive. FIG. 2A also illustrates empty space 210 as part of third layer 206 where there is no adhesive. Fourth layer 208 may be a release liner that is the same or similar to fourth layer 108 as shown and described in FIG. 1.

FIG. 2B illustrates a cross-sectional view of an example of a self-adhesive absorbent patch 220 having a four-layer configuration and two strips of adhesive in third layer 226. First layer 222 may face away from the body when applied to a user. First layer 222 may be the same or similar to first layer 102 as shown and described in FIG. 1. First layer 222 may be bonded and/or adhered and/or laminated to a second layer 224 that may be a fluid, liquid, and/or moisture resistant and/or impermeable layer similar to second layer 104 as shown and described in FIG. 1. Third layer 226 may be a non-continuous layer of adhesive consisting of, for example, two strips of adhesive. FIG. 2A also illustrates the empty space 230 as part of third layer 226 where there is no adhesive. Fourth layer 228 may be a release liner that is the same or similar to fourth layer 108 as shown and described in FIG. 1.

FIG. 2C illustrates a cross-sectional view of an example of a self-adhesive absorbent patch 240 having a three-layer configuration and three strips of adhesive in second layer 246. First layer 242 may face away from the body when applied to a user. First layer 242 may be the same or similar to first layer 102 as shown and described in FIG. 1. First layer 242 may be bonded and/or adhered and/or laminated to a second layer 246 that may be a non-continuous layer of adhesive consisting of, for example, three strips of adhesive. FIG. 2A also illustrates the empty space 250 as part of third layer 246 where there is no adhesive. Fourth layer 248 may be a release liner that is the same or similar to fourth layer 108 as shown and described in FIG. 1.

FIG. 2D illustrates a cross-sectional view of an example of a self-adhesive absorbent patch 260 having a three-layer configuration and two strips of adhesive in second layer 266. First layer 262 may face away from the body when applied to a user. First layer 262 may be the same or similar to first layer 102 as shown and described in FIG. 1. First layer 262 may be bonded and/or adhered and/or laminated to a second layer 266 that may be a non-continuous layer of adhesive consisting of, for example, two strips of adhesive. FIG. 2A also illustrates the empty space 270 as part of third layer 266 where there is no adhesive. Fourth layer 268 may be a release liner that is the same or similar to fourth layer 108 as shown and described in FIG. 1.

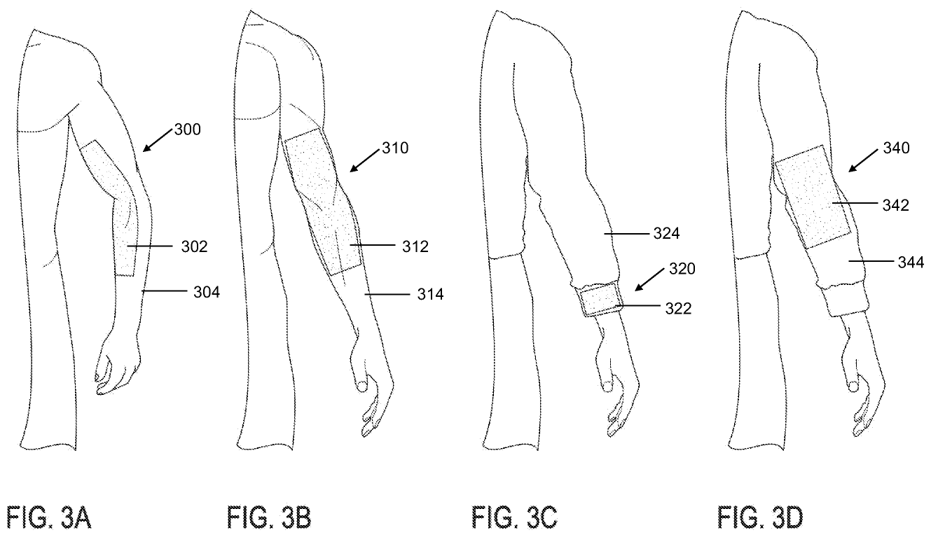

FIGS. 3A, 3B, 3C, and 3D illustrate example placements of a self-adhesive absorbent patch on a user's body and/or on a garment in a similar location on the user's body. FIGS. 3A and 3B, illustrate example placement of a self-adhesive absorbent patch on a user's body. FIGS. 3C and 3D illustrate example placement of a self-adhesive absorbent patch on a user's long-sleeve garment. Depending upon the application, other placement locations may be contemplated. For example, if a user wears a neck gaiter while performing a winter activity outdoors, a self-adhesive absorbent patch may be applied to the inside of the neck gaiter between the neck gaiter and the user's mouth to absorb droplets and prevent droplets from being expelled through the neck gaiter, which may comprise a thin non-absorbent material. Other similar example also may be used.

FIG. 3A illustrates the example placement 300 of self-adhesive absorbent patch 302 on user's arm 304. In this example placement 300, the user's elbow as depicted is directed slightly away from the body, so self-adhesive absorbent patch 302 is shown from the side as applied. In this example placement 300, self-adhesive absorbent patch 302 may be placed on a user's arm 304, with the midpoint of self-adhesive absorbent patch 302 placed approximately where the user's arm bends at the elbow, such that self-adhesive absorbent patch 302 covers part of the user's forearm and part of the user's upper arm. In this example placement 300, the self-adhesive absorbent patch is roughly rectangular in shape.

FIG. 3B illustrates the example placement 310 of self-adhesive absorbent patch 312 on user's arm 314. In this example placement 310, the user's elbow as depicted is facing the same direction as the back of the body, so self-adhesive absorbent patch 312 is shown from the front as applied. In this example placement 310, self-adhesive absorbent patch 312 is placed on user's arm 314, with the midpoint of self-adhesive absorbent patch 312 placed approximately where the user's arm bends at the elbow, such that the self-adhesive absorbent patch 312 covers part of the user's forearm and part of the user's upper arm. In this example placement 310, the self-adhesive absorbent patch is roughly rectangular in shape.

FIG. 3C illustrates the example placement 320 of self-adhesive absorbent patch 322 on the wrist of user's garment 324. In this example placement 320, self-adhesive absorbent patch 322 is placed such that self-adhesive absorbent patch 322 wraps around the wrist of garment 324 and is located on the part of the wrist that would be closest to a user's face when the user brings the wrist up towards the user's face. In this example placement 320 the self-adhesive absorbent patch is rectangular in shape.

FIG. 3D illustrates the example placement 340 of self-adhesive absorbent patch 342 on user's garment 344. In this example placement 340, the user's elbow as depicted is facing the same direction as the back of the body, so self-adhesive absorbent patch 342 is shown from the front as applied. In this example placement 340, self-adhesive absorbent patch 342 is placed on a user's arm, with the midpoint of self-adhesive absorbent patch 342 placed approximately where the user's arm bends at the elbow, such that self-adhesive absorbent patch 342 covers part of the user's forearm and part of the user's upper arm. In this example placement 340 the self-adhesive absorbent patch is rectangular in shape.

FIGS. 4A, 4B, 4C, 4D, 4E, 4F, 4G, 4H, 4I, 4J, 4K illustrate various example shapes of a self-adhesive patch and patterns of adhesive that may be applied on a self-adhesive absorbent patch. These examples are not intended to exclude other shapes of a self-adhesive absorbent patch and/or shapes or patterns of adhesive that may be used on a self-adhesive absorbent patch.

FIGS. 4A, 4B, 4C, and 4F illustrate various examples of a rectangular self-adhesive absorbent patch with adhesive strips parallel to the longest sides of the self-adhesive absorbent patch. FIGS. 4D and 4H illustrate various examples of a rectangular self-adhesive absorbent patch with adhesive strips parallel to the shortest sides of the self-adhesive absorbent patch. FIGS. 4F and 4G illustrate various examples of an oval-shaped self-adhesive absorbent patch. FIG. 4I illustrates a self-adhesive absorbent patch with adhesive around the entire perimeter of the self-adhesive absorbent patch. FIGS. 4J and 4K illustrate various examples of a rectangular self-adhesive absorbent patch with spots of adhesive. FIG. 4E illustrates an example of a self-adhesive absorbent patch with adhesive covering the entire back.

FIG. 4A illustrates an example of a rectangular self-adhesive absorbent patch 400 with adhesive strip 404 in the center of self-adhesive absorbent patch 400 that is parallel to longest sides 406 of self-adhesive absorbent patch patch 400. As shown in FIG. 4A, self-adhesive absorbent patch 400 may include one or more empty spaces and/or voids 402 that may be located within and/or created by adhesive layer 404.

FIG. 4B illustrates an example of a rectangular self-adhesive absorbent patch 410 with three adhesive strips 414 that are parallel to the longest sides 416 of self-adhesive absorbent patch 410. As shown in FIG. 4B, self-adhesive absorbent patch 410 may include one or more empty spaces 412 that may be located within and/or created by adhesive layer 414.

FIG. 4C illustrates an example of a rectangular self-adhesive absorbent patch 420 with two adhesive strips 424 that are parallel to the longest sides 426 of self-adhesive absorbent patch 420. As shown in FIG. 4C, self-adhesive absorbent patch 420 may include one or more empty spaces 422 that may be located within and/or created by adhesive layer 424.

FIG. 4D illustrates an example of a rectangular self-adhesive absorbent patch 430 with three adhesive strips 434 that are parallel to the shortest sides 438 of self-adhesive absorbent patch 430. As shown in FIG. 4D, self-adhesive absorbent patch 430 may include one or more empty spaces 432 that may be located within and/or created by adhesive layer 434.

FIG. 4E illustrates an example of a rectangular self-adhesive absorbent patch 440 with adhesive 444 on the entire face of self-adhesive absorbent patch 440.

FIG. 4F illustrates an example of an oval self-adhesive absorbent patch 450 with two adhesive strips 454. As shown in FIG. 4F, self-adhesive absorbent patch 450 may include one or more empty spaces 452 that may be located within and/or created by adhesive layer 454.

FIG. 4G illustrates an example of an oval self-adhesive absorbent patch 460 with one adhesive strip 464 placed in the center of self-adhesive absorbent patch 460. As shown in FIG. 4G, self-adhesive absorbent patch 460 may include one or more empty spaces 462 that may be located within and/or created by adhesive layer 464.

FIG. 4H illustrates an example of a rectangular self-adhesive absorbent patch 470 with three adhesive strips 474 that are parallel to the shortest sides 478 of self-adhesive absorbent patch 470. Two of the adhesive strips 474 are flush with the shortest sides 478 of self-adhesive absorbent patch 470. As shown in FIG. 4H, self-adhesive absorbent patch 470 may include one or more empty spaces 472 that may be located within and/or created by adhesive layer 474. As shown in FIG. 4H, self-adhesive absorbent patch 470 may have adhesive strips 474 along one or more of its edges.

FIG. 4I illustrates an example of a rectangular self-adhesive absorbent patch 480 with adhesive 484 around the perimeter of self-adhesive absorbent patch 480. As shown in FIG. 4I, self-adhesive absorbent patch 480 may include one or more empty spaces 482 that may be located within and/or created by adhesive layer 484.

FIG. 4J illustrates an example of a rectangular self-adhesive absorbent patch 490 with adhesive rectangles 494 placed in the center and corners of the self-adhesive absorbent patch 490. As shown in FIG. 4J, self-adhesive absorbent patch 490 may include one or more empty spaces 492 that may be located within and/or created by adhesive layer 494. In this example, adhesive 494 is placed to secure the corners and center of self-adhesive absorbent patch 490.

FIG. 4K illustrates an example of a rectangular self-adhesive absorbent patch 495 with adhesive circles and/or dots 498 placed in the center and corners of the self-adhesive absorbent patch 495. As shown in FIG. 4K, a self-adhesive absorbent patch 495 may include one or more empty spaces 496 that may be located within and/or created by an adhesive layer 498. In this example, adhesive 498 is placed to secure the corners and central area of the self-adhesive absorbent patch 495.

It will be appreciated that any number of adhesive strips, dots, rectangles, and/or other shapes and/or configurations may be used to cause self-adhesive absorbent patch to adhere to a user and/or a user garment, even if not specifically shown in these examples. For example, strips of adhesive may be placed closer to or farther away from the edges of the self-adhesive absorbent patch, placed on the diagonal, and/or many other configurations. Additionally, for example, strips of adhesive may be narrower or wider than those shown in the illustrations herein. It will also be appreciated that while rectangular and oval shapes are depicted here, a self-adhesive absorbent patch may take any number of shapes, including another type of polygon or non-polygon, depending on things such as the placement on the body, season, type of clothing worn by the user, and other relevant factors.

The detailed description above sets forth numerous specific details to provide a thorough understanding of the invention. However, those skilled in the art will appreciate that the invention may be practiced without these specific details.

The terms and expressions that have been used in the foregoing specification are used as terms of description and not of limitation. By using those terms and expressions, there is no intention to exclude any equivalent terms and/or x expressions.

It should be understood by those skilled in the art that various modifications, combinations, sub-combinations, and alterations may occur depending on design requirements and other factors insofar as they are within the scope of the appended claims and the equivalents thereof.

Accordingly, the embodiments of the present inventions are not to be limited in scope by the specific embodiments described herein. Further, although some of the embodiments of the present disclosure have been described herein in the context of a particular implementation in a particular environment for a particular purpose, those of ordinary skill in the art should recognize that its usefulness is not limited thereto and that the embodiments of the present inventions can be beneficially implemented in any number of environments for any number of purposes. Accordingly, the claims set forth below should be construed in view of the full breadth and spirit of the embodiments of the present inventions as disclosed herein. While the foregoing description includes many details and specificities, it is to be understood that these have been included for purposes of explanation only, and are not to be interpreted as limitations of the invention. Many modifications to the embodiments described above can be made without departing from the spirit and scope of the invention.

What is claimed is:

1. A self-adhesive absorbent patch, comprising:
a first layer that is an outermost layer when the self-adhesive absorbent patch is applied to a user's body and/or garment and absorbs liquids, droplets, and/or moisture expelled by the user;
a second layer that is bonded to the first layer and is resistant or impermeable so as to prevent or limit the liquids, droplets, and/or moisture absorbed from the first layer from being deposited onto the user's body and/or garment;
a third layer comprised of a double-sided adhesive, wherein a first side of the double-sided adhesive adheres to the second layer and a second side of the double-sided adhesive adheres to the user's body and/or garment when the user applies the self-adhesive absorbent patch to the user's body and/or garment; and
a fourth layer that is removably attached to the third layer before the user applies the self-adhesive absorbent patch to the user's body and/or garment.

2. The self-adhesive absorbent patch of claim 1, wherein:
the first layer is an absorbent layer comprised of cotton, cellulose, polyester, viscose, and/or other natural and/or synthetic fibers that absorb liquids, droplets, and/or moisture expelled by the user.

3. The self-adhesive absorbent patch of claim 1, wherein:
the second layer is comprised of polyurethane and/or polyethylene.

4. The self-adhesive absorbent patch of claim 1, wherein:
the third layer is comprised of acrylic, acrylate, and/or silicon.

5. The self-adhesive absorbent patch of claim 4, wherein:
the double-sided adhesive in the third layer is disposed in the third layer such that it forms one or more empty areas in the third layer that are devoid of the double-sided adhesive.

6. The self-adhesive absorbent patch of claim 1, wherein:
the fourth layer is a release liner that adheres to the third layer before the user applies the self-adhesive absorbent patch to the user's body and/or garment and is removed from the third layer to expose the second side of the double-sided adhesive so that the user can apply the second side to the user's body and/or garment.

7. The self-adhesive absorbent patch of claim 1, wherein:
after the self-adhesive absorbent patch is applied to the user's body and/or garment, the second side of the double-sided adhesive is capable of being separated from the user's body and/or garment without adversely affecting the user's body and/or the garment.

8. A self-adhesive absorbent patch, comprising:
a first layer that is an outermost layer when the self-adhesive absorbent patch is applied to a user's body and/or garment and absorbs liquids, droplets, and/or moisture expelled by the user;
a second layer comprised of a double-sided adhesive, wherein a first side of the double-sided adhesive adheres to the second layer and a second side of the double-sided adhesive adheres to the user's body and/or garment when the user applies the self-adhesive absorbent patch to the user's body and/or garment; and
a third layer that is removably attached to the third layer before the user applies the self-adhesive absorbent patch to the user's body and/or garment.

9. The self-adhesive absorbent patch of claim 8, wherein:
the first layer is an absorbent layer comprised of cotton, cellulose, polyester, viscose, and/or other natural and/or synthetic fibers that absorb liquids, droplets, and/or moisture expelled by the user.

10. The self-adhesive absorbent patch of claim 8, wherein:
the second layer is comprised of acrylic, acrylate, and/or silicon.

11. The self-adhesive absorbent patch of claim 10, wherein:
the double-sided adhesive in the second layer is disposed in the third layer such that it forms one or more empty areas in the second layer that are devoid of the double-sided adhesive.

12. The self-adhesive absorbent patch of claim 8, wherein:
the third layer is a release liner that adheres to the third layer before the user applies the self-adhesive absorbent patch to the user's body and/or garment and is removed from the third layer to expose the second side of the double-sided adhesive so that the user can apply the second side to the user's body and/or garment.

13. The self-adhesive absorbent patch of claim 14, wherein:
after the self-adhesive absorbent patch is applied to the user's body and/or garment, the second side of the double-sided adhesive is capable of being separated from the user's body and/or garment without adversely affecting the user's body and/or the garment.

14. A self-adhesive absorbent patch, comprising:
an absorbent layer that is an outermost layer when the self-adhesive absorbent patch is applied to a user's body and/or garment and absorbs liquids, droplets, and/or moisture expelled by the user;
an impermeable layer that is bonded to the first layer and resists or prevents the liquids, droplets, and/or moisture absorbed from the absorbent layer from being deposited onto the user's body and/or garment;
an adhesive layer comprised of a double-sided adhesive, wherein a first side of the double-sided adhesive adheres to the impermeable layer and a second side of the double-sided adhesive adheres to the user's body and/or garment when the user applies the self-adhesive absorbent patch to the user's body and/or garment; and
a release liner that is removably attached to the adhesive layer before the user applies the self-adhesive absorbent patch to the user's body and/or garment.

15. The self-adhesive absorbent patch of claim 14 wherein:
the absorbent is layer comprised of cotton, cellulose, polyester, viscose, and/or other natural and synthetic fibers that absorb liquids, droplets, and/or moisture expelled by the user.

16. The self-adhesive absorbent patch of claim 14, wherein:
the resistant or impermeable layer is comprised of polyurethane and/or polyethylene.

17. The self-adhesive absorbent patch of claim 14, wherein:
the adhesive layer is comprised of acrylic, acrylate, and/or silicon.

18. The self-adhesive absorbent patch of claim 17, wherein:
the double-sided adhesive in the adhesive layer is disposed in the third layer such that it forms one or more empty areas in the third layer that are devoid of the double-sided adhesive.

19. The self-adhesive absorbent patch of claim 14, wherein:
the release liner adheres to the adhesive layer before the user applies the self-adhesive absorbent patch to the user's body and/or garment and is removed from the adhesive layer to expose the second side of the double-sided adhesive so that the user can apply the second side to the user's body and/or garment.

20. The self-adhesive absorbent patch of claim 14, wherein:
after the self-adhesive absorbent patch is applied to the user's body and/or garment, the second side of the double-sided adhesive is capable of being separated from the user's body and/or garment without adversely affecting the user's body and/or the garment.
